(19) United States
(12) Patent Application Publication
(10) Pub. No.: US 2006/0122164 A1
(43) Pub. Date: Jun. 8, 2006

(54) Carboxylic acid esters of pharmaceutical compounds

(76) Inventors: Gerd Ascher, Kundl (AT); Klaus Thirring, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/541,017

(22) PCT Filed: Jan. 27, 2004
(86) PCT No.: PCT/EP04/00683

§ 371 Date: Jun. 28, 2005

(30) Foreign Application Priority Data
Jan. 28, 2003 (GB) 0301938.7

(51) Int. Cl. A61K 31/545; A61K 31/43; C07D 501/14; C07D 499/00

(52) U.S. Cl. 514/192; 514/202; 540/222; 540/310

(57) ABSTRACT

A pharmaceutically active compound having a carboxylic acid group —COOH as a part of its chemical structure which —COOH group is in the form of a carboxylic acid ester.

The present invention relates to carboxylic acid esters of pharmaceutical compounds, e.g. β-lactam antibiotics, such as cephalosporins.

In one aspect the present invention provides a pharmaceutically active compound having a carboxylic acid group —COOH as a part of its chemical structure which —COOH group is in the form of a carboxylic acid ester and which carboxylic acid ester is selected from the group consisting of
1-(2,3-disubstituted 1-propoxycarbonyloxy)-ethyl carboxylic acid ester, wherein the substituents are selected from the group consisting of hydroxy and (C₁₋₂₂)alkylcarbonyloxy,
1-(1,3-disubstituted 2-propoxycarbonyloxy)-ethyl carboxylic acid ester, wherein the substituents are selected from the group consisting of hydroxy and (C₁₋₂₂)alkylcarbonyloxy,
1-(9H-fluorene-9-yl-(C₁₋₄)alkanyloxycarbonyloxy)-ethyl carboxylic acid ester,
1-(decahydro-naphthalene-2-yl-oxycarbonyloxy)-ethyl carboxylic acid ester and
1-(2-amino(C₁₋₆)alkoxycarbonyloxy)-ethyl carboxylic acid ester,
   with the proviso that, if the pharmaceutically active compound is a penicillin, then, 1-(2-amino(C₁₋₄)alkoxycarbonyloxy)-ethyl carboxylic acid esters are excluded.

A pharmaceutically active compound of the present Invention includes β-lactam antibiotics, such as cephalosporins and penicillins, e.g. compounds comprising the basic structural elements of groups of formula 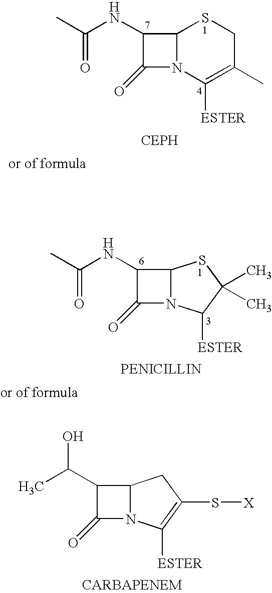
wherein X is a substituted pyrrolidinyl or substituted amino(C₁₋₄)alkyl, e.g. substituted by NH═CH—, and ESTER is selected from the group consisting of
1-(2,3-disubstituted 1-propoxycarbonyloxy)-ethyl carboxylic acid ester, wherein the substituents are selected from the group consisting of hydroxy and (C₁₋₂₂)alkylcarbonyloxy,
1-(1,3-disubstituted 2-propoxycarbonyloxy)-ethyl carboxylic acid ester, wherein the substituents are selected from the group consiting of hydroxy and (C₁₋₂₂)alkylcarbonyloxy,
1-(9H-fluorene-9-yl-(C₁₋₄)alkanyloxycarbonyloxy)-ethyl carboxylic acid ester,
1-(decahydro-naphthalene-2-yl-oxycarbonyloxy)-ethyl carboxylic acid ester and
1-(2-amino(C₁₋₆)alkoxycarbonyloxy)-ethyl carboxylic acid ester,
   with the proviso that, if the pharmaceutically active compound is a penicillin, then compounds, wherein ESTER is 1-(2-amino(C₁₋₄)alkoxycarbonyloxy)-ethyl-oxy-carbonyl, are excluded.

In another aspect the present invention provides a pharmaceutically active compound of the present invention, which is a pharmaceutically active β-lactam, e.g. of formula CEPH, PENICILLIN or CARBAPENEM, wherein ESTER are as defined above, with the proviso that, if the pharmaceutically active compound is a penicillin, then compounds, wherein ESTER is 1-(2-amino(C₁₋₄)alkoxycarbonyloxy)-ethyl-oxy-carbonyl, are excluded.

In another aspect the present invention provides a pharmaceutically active compound of the present invention, which is a cephalosporin, e.g. comprising the basic structural elements as set out in formula CEPH, wherein ESTER are as defined above.

In another aspect the present invention provides a pharmaceutically active compound of the present invention, which is a penicillin, e.g. comprising the basic structural elements as set out in formula PENICILLIN and ESTER are as defined above, with the proviso that, if the pharmaceutically active compound is a penicillin, then compounds, wherein ESTER is 1-(2-amino(C₁₋₄)alkoxycarbonyloxy)-ethyl-oxy-carbonyl, are excluded.

In another aspect the present invention provides a pharmaceutically active compound of the present invention, which is a β-lactam, e.g. comprising the basic structural elements as set out in formula CARBAPENEM and ESTER are as defined above,

In another aspect the present invention provides a pharmaceutically active compound of the present invention, wherein the —COOH group is in the form of an ester, e.g. a group ESTER, which is of formula 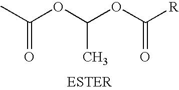
wherein R is selected from the group consisting of
disubstituted 1-propoxy or 2-propoxy substituted with OH or (C₁₋₂₂)alkylcarbonyloxy,
9H-fluorene-9-yl-(C₁₋₄)alkoxy,
decahydronaphthoxy and
amino(C₁₋₆)alkoxy,
   with the proviso that, if the pharmaceutically active compound is a penicillin, then compounds, wherein ESTER is 1-(2-amino(C₁₋₄)alkoxycarbonyloxy)-ethyl-oxy-carbonyl, are excluded.

In another aspect the present invention provides a pharmaceutically active compound of the present invention, wherein the —COOH group is in the form of an ester, e.g. a group ESTER, selected from the group consisting of
1-(2,3-dihydroxy-1-propoxycarbonyloxy)-ethyl-oxy-carbonyl,
1-(2,3-diacetoxy-1-propoxycarbonyloxy)-ethyl-oxy-carbonyl,
1-(1,3-diacetoxy-2-propoxycarbonyloxyyethyl-oxy-carbonyl,
1-(2-octanoyloxy-3-acetoxy-1-propoxycarbonyloxy)-ethyl-oxy-carbonyl,
1-(2-acetoxy-3-octanoyloxy-1-propoxycarbonyloxy)-ethyl-oxy-carbonyl,
1-(9H-fluorene-9-yl-methoxycarbonyloxy)-ethyl-oxy-carbonyl,
1-(9H-fluorene-9-yl-ethoxycarbonyloxyyethyl-oxy-carbonyl,
1-(decahydro-naphthalene-2-yl-oxycarbonyloxy)-ethyl-oxy-carbonyl and
1-(2-amino-ethoxycarbonyloxy)-ethyl-oxy-carbonyl
   with the proviso that, if the pharmaceutically active compound is a penicillin, then compounds, wherein ESTER is 1-(2-amino(C₁₋₄)alkoxycarbonyloxy)-ethyl-oxy-carbonyl, are excluded.

In another aspect the present Invention provides a pharmaceutically active compound of the present invention, wherein the —COOH group is in the form of an ester, e.g. a group ESTER, selected from the group consisting of
1-(2,3-disubstituted 1-propoxycarbonyloxy)-ethyl carboxylic acid ester, wherein the substituents are selected from the group consisting of hydroxy and (C₁₋₂₂)alkylcarbonyloxy,
1-(1,3-disubstituted 2-propoxycarbonyloxy)-ethyl carboxylic acid ester, wherein the substituents are selected from the group consisting of hydroxy and (C₁₋₂₂)alkylcarbonyloxy,
1-(9H-fluorene-9-yl-(C₁₋₄)alkanyloxycarbonyloxy)-ethyl carboxylic acid ester, and
1-(decahydro-naphthalene-2-yl-oxycarbonyloxy)-ethyl carboxylic acid ester.

In another aspect the present invention provides a pharmaceutically active compound of the present invention of formula CEPH selected from the group consisting of
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-{[3-ethyl-2-(methylimino)-imidazolidin-1-yl]-hydrazonomethyl}-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-[(carbamimidoyl)-hydrazonomethyl]-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-[(1,4,5,6,-tetrahydropyrimidin-2-yl)-hydrazonomethyl]-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-{methyl-[N-(4-amino-cyclohexyl)-carbamimidoyl]-hydrazonomethyl}-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-[(2-dimethylamino-4,5-dihydro-imidazol-1-yl-imino)-methyl]-8-oxo-5-thia-1-aza-bicyclo[4.2.0]oct-2-ene-2-carboxylic acid,
3-Carbamoyloxymethyl-7-{2-furan-2-yl-[(Z)-methoxyimino]-acetylamino}-8-oxo-5-thia-1-aza-bicyclo[4.2.0]oct-2-ene-2-carboxylic acid, and
7-{2-(5-Amino-[1,2,4]thiadiazol-3-yl)-2-(fluoromethoxyimino)-acetylamino}-3-[(3-ethyl-2-(methylimino)-imidazol-1-yl-imino)-methyl]-8-oxo-5-thia-1-aza-bicyclo[4.2.0]oct-2-ene-2-carboxylic acid,

In another aspect the present invention provides a pharmaceutically active compound of the present invention selected from the group consisting of
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,3-diacetoxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(1,3-diacetoxy-2-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,-octanoyl-3-acetoxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2-acetoxy-3-octanoyl-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2-aminoethoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(decahydro-naphthalen-2-yl-oxycarbonyloxy)-ethyl ester,
7-{2-(5-Amino-[1,2,4]thiadiazol-3-yl)-2-(fluoromethoxyimino)-acetylamino}-3-[(imino-piperazin-1-yl-methyl)-hydrazonomethyl]-8-oxo-5-thia-1-aza-bicyclo[4.2.0]oct-2-ene-2-carboxylic acid 1-(2-aminoethoxycarbonyloxy)-ethyl ester,
7-{2-(5-Amino-[1,2,4]thiadiazol-3-yl)-2-(fluoromethoxyimino)-acetylamino}-3-[(imino-piperazin-1-yl-methyl)-hydrazonomethyl]-8-oxo-5-thia-1-aza-bicyclo[4.2.0]oct-2-ene-2-carboxylic acid 1-(decahydro-naphthalen-2-yl-oxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-([1-(9H-fluoren-9-yl)-ethoxycarbonyloxy)-ethyl ester,
7-{2-(5-Amino-[1,2,4]thiadiazol-3-yl)-2-(fluoromethoxyimino)-acetylamino}-3-[{[3-ethyl-2-(methylimino)-imidazolidin-1-yl]-hydrazonomethyl}-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-[carbamimidoyl)-hydrazonomethyl]-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-[(1,4,5,6,-tetrahydropyrimidin-2-yl)-hydrazonomethyl]-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-{methyl-[N-(4-amino-cyclohexyl)-carbamimidoyl]-hydrazonomethyl}-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-[(2-dimethylamino-4,5-dihydro-imidazol-1-yl-imino)-methyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[(carbamimidoyl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[(1,4,5,6,-tetrahydropyrimidin-2-yl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[(2-dimethylamino-4,5-dihydro-imidazol-1-yl-imino)-methyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-{[3-ethyl-2-(methylimino)-imidazolidin-1-yl]-hydrazonomethyl}-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-{methyl-[N-(4-amino-cyclohexyl)-carbamimidoyl]-hydrazonomethyl}-8-oxo-5-thia-1-aza-bicyclo[4.2.0]oct-2-ene-2-carboxylic acid 1-(2,3-dihydroxy-1-propoxycarbonyloxy)-ethyl ester,
3-Carbamoyloxymethyl-7-{2-furan-2-yl-[(Z)-methoxyimino]-acetylamino}-8-oxo-5-thia-1-aza-bicyclo[4.2.0]oct-2-ene-2-carboxylic acid 1-(9H-fluoren-9-yl-methoxycarbonyloxy)-ethyl ester, and
3-Carbamoyloxymethyl-7-{2-furan-2-yl-[(Z)-methoxyimino]-acetylamino}-8-oxo-5-thia-1-aza-bicyclo[4.2.0]oct-2-ene-2-carboxylic acid 1-(2,3-dihydroxy-1-propoxycarbonyloxy)-ethyl ester,

In another aspect the present invention provides a pharmaceutically active compound of the present invention, which is a compound of formula 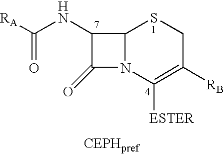
wherein
ESTER is as defined above,
R_{A} is a group of formula
   a. 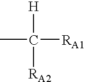
   wherein
R_{A1} is unsubstituted or one- or morefold substituted
(C₆₋₁₈)aryl, e.g. phenyl,
(C₆₋₁₈)aryloxy, e.g. phenoxy,
(C₅₋₆)cycloalkenyl-(C₁₋₄)alkyl,
   and
R_{A2} is
hydroxy, (C₁₋₄)alkyl, formyloxy, methyleneamino, azido,
(C₆₋₁₈)aryl, e.g. phenyl,
unsubstituted or substituted amino, e.g. heterocyclyl-amino, (C₃₋₆)cycloalkylcarbonylamino, e.g. wherein cycloalkyl optionally is substituted by oxo, SO₃H or carboxyl;
   or

b. —(CH₂)ₙ—R_{A3}
wherein R_{A3} is
S—R_{A4}, wherein R_{A4} is heterocyclyl having 5 or 6 ring members and 1 to 4 heteroatoms selected from N, O, S, e.g. pyridinyl, (C₁₋₄)alkyl or (C₂₋₄)alkenyl, which alkyl or alkenyl is optionally substituted by carboxyl, cyano, amino,
(C₁₋₄)alkyl, which alkyl is optionally substituted by carboxyl, amino,
heterocyclyl having 5 or 6 ring members and 1 to 4 heteroatoms selected from N, O, S, e.g. thiophenyl, 1-H-tetrazolyl, isoxazolyl, 1H-pyridin-4-on-1-yl, piperazinyl, which heterocyclyl is optionally substituted by (C₁₋₄)alkyl, amino, phenyl, oxo, halogen, carboxyl,
(C₆₋₁₈)aryl, e.g. phenyl,
(C₆₋₁₈)aryloxy, e.g. phenoxy,
   and
n is 0 to 6, e.g. o or 1;
   or
   c. 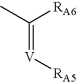
   wherein
V is N or CH,
R_{A6} is heterocyclyl having 5 or 6 ring members and 1 to 4 heteroatoms selected from N, O, S,
R_{A5} is
hydroxy,
(C₁₋₄)alkyl, which alkyl is optionally substituted by carboxyl,
(C₁₋₄)alkoxy, which alkoxy is optionally substituted by carboxyl, halogen, e.g. fluoro;
   or
   d. 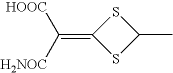
   and

R_{B} is a hydrogen, hydroxyl, halogen, e.g. chloro, (C₁₋₄)alkoxy or a group of formula

a. —(CH₂)ₘ—R_{B1}
wherein R_{B1} is
hydrogen, halogen or hydroxy,
(C₁₋₄)alkoxy,
(C₁₋₄)alkylcarbonyloxy, (C₂₋₄)alkenylcarbonyloxy, which alkyl- or alkenylcarbonyloxy is optionally subtituted by (C₆₋₁₈)aryl, e.g. phenyl or (C₁₋₄)alkoxy,
(C₁₋₄)alkoxycarbonyl,
aminocarbonyloxy,
heterocyclyl having 5 or 6 ring members, and 1 to 4 hetereoatoms selected from N, O, S including heterocyclyl anellated with another ring (system) or bridged heterocylcyl, or
S—R_{B1′}, wherein R_{B1′} is heterocyclyl having 5 or 6 ring members and 1 to 4 hetereoatoms selected from N, O, S,
   and
m is 1 to 4, e.g. 1;
   or

b. —C═C—R_{B2}
wherein R_{B2} is
hydrogen,
(C₁₋₄)alkyl, optionally substituted by (di(C₁₋₄)alkyl)(aminocarbonyl(C₁₋₄)alkyl)ammonium with an appropriate anion, e.g. chloride, sulfate, nitrate, tetrafluoroborate or is present as an inner salt,
(C₆₋₁₈)aryl, such as phenyl, optionally substituted ny nitro, or
heterocyclyl having 5 to 6 ring members and 1 to 4 hetereoatoms selected from N, O, S;
   or
   c. 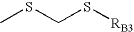
   wherein R_{B3} is heterocyclyl having 5 or 6 ring members and 1 to 4 hetereatoms selected from N, O, S, e.g. a triazolyl;
   or
   d. 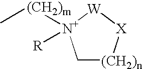
optionally anellated with another ring (system), wherein
X, Y and W independently of each other are C, CH, CH₂ or N, which ring is optionally substituted by aminocarbonyl, amino, hydroxyl(C₁₋₄)alkyl,
R is not present or is present and is (C₁₋₄)alkyl, m is 0 or 1 and n is 1 or 2.

“Heterocyclyl” includes heterocyclyl having 5 or 6 ring members and 1 to 4 heteroatoms selected from N, O, S, which may be wholly or partly saturated, e.g. at least one N, which heterocyclyl is optionally anellated with another ring (system), e.g. wherein substituents are selected from hydroxyl, (C₁₋₄)alkyl, (C₁₋₄)alkoxy, amino(C₁₋₄)alkyl, aminocarbonyl, (C₁₋₄)alkoxyimino, imino(C₁₋₄)oxycarbonyloxy(C₁₋₄)alkyl, imino(C₁₋₄)oxyalkyl or iminohalo-(C₁₋₄)alkyl.

Heterocyclyl preferably is pyrrolyl, imidazolyl, benzimidazolyl, pyrazolyl, pyridyl, pyrazinyl, pyrimidinyl, pyridazinyl, indolyl, oxazolyl, thiophenyl, azolyl, thiazolyl, triazolyl, benzothiophenyl, furanyl, and tetrazolyl; and may be unsubstituted or substituted by one or more, especially one or two, substitutents selected from the group as indicated above.

In another aspect the present invention provides a pharmaceutically active compound of the present invention which is a compound of formula 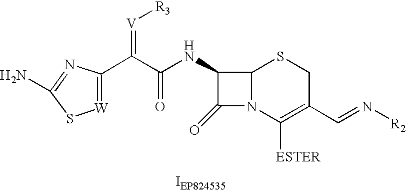
wherein
ESTER is as defined above,
R₂ denotes a group of formula 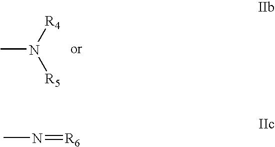
   wherein
R₄ denotes hydrogen, (C₁₋₈)alkyl, (C₂₋₈)alkenyl, (C₃₋₆)cycloalkyl, phenyl, (C₁₋₁₂)acyl or heterocyclyl,
R₅ denotes hydrogen, (C₁₋₈)alkyl, (C₂₋₈)alkenyl, (C₃₋₆)cycloalkyl, phenyl or a group of formula 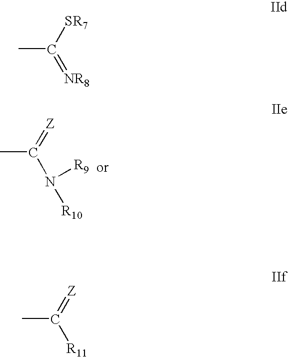
   wherein
R₇ denotes (C₁₋₈)alkyl or phenyl,
R₈ denotes hydrogen, (C₃₋₆)cycloalkyl or (C₁₋₈)alkyl,
R₉ denotes hydrogen or (C₁₋₈)alkyl,
R₁₀ denotes hydrogen, (C₁₋₈)alkyl, hydroxyl, amino, phenyl, (C₂₋₈)alkenyl, (C₃₋₆)cycloalkyl, heterocyclyl, or a group of formula —N═CH—Phe, wherein Phe denotes phenyl,
R₉ and R₁₀ together with the nitrogen atom denote heterocyclyl,
Z denotes oxygen, sulphur, or N—R₃, wherein
R₁₃ denotes hydrogen, (C₁₋₈)alkyl or (C₃₋₆)cycloalkyl,
R₁₁ denotes hydrogen, (C₁₋₈)alkyl, phenyl, (C₃₋₆)cycloalkyl or heterocyclyl; or
R₄ and R₅ together with the nitrogen denote heterocyclyl,
R₆ denotes heterocyclyl,
W denotes N or CH,
V denotes CH or NO, and
R₃ denotes hydrogen, (C₁₋₈)alkyl, halo(C₁₋₄)alkyl, (C₁₋₁₂)acyl or carboxyl.

In another aspect the present invention provides a pharmaceutically active compound of the present invention which is a compound of formula 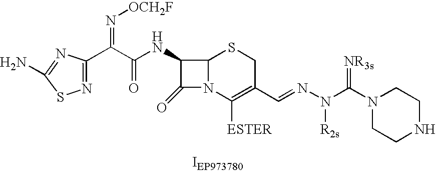
wherein
ESTER is as defined above,
R_{2S} denotes (C₁₋₆)alkyl, ar(C₁₋₆)alkyl, (C₂₋₆)alkenyl or (C₂₋₈)alkinyl,

R_{3S} denotes hydrogen, (C₁₋₆)alkyl, ar(C₁₋₆)alkyl, (C₂₋₆)alkenyl, (C₂₋₈)alkinyl or (C₃₋₈)cycloalkyl.

In another aspect the present invention provides a pharmaceutically active compound of the present invention which is a compound of formula 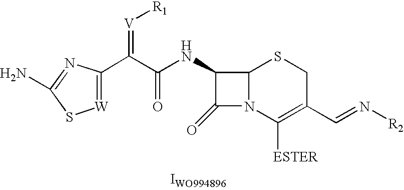
wherein
ESTER is as defined above,
W denotes CH or N,
V denotes CH or NO,
R₁ denotes hydrogen, (C₁₋₁₂)acyl, carboxyl, alkyl or haloalkyl,
R₂ denotes a group of formula 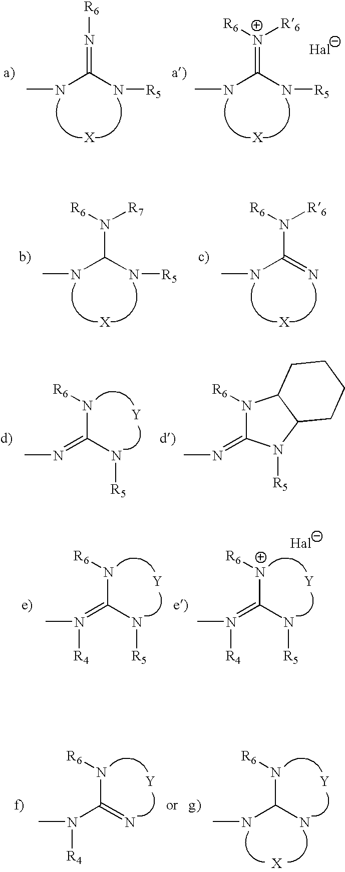
   wherein
X and Y independently of each other each denote (C₂₋₅)alkylene, or (C₂₋₅)alkenylene wherein one —C═C— double bond is present, or, in case of at least C₄-alkenylene, wherein two —C═C— double bonds are present,
R₄ denotes hydrogen or alkyl,
R₅ denotes hydrogen, alkyl, or aminoiminomethyl,
R₆ denotes hydrogen, alkyl, cycloalkyl, amino, hydroxy, alkoxy, heterocyclyl or a group of formula —N═CHR₈, wherein R₈ denotes alkyl, aryl or heterocyclyl, or
R₅ and R₆ together with the nitrogen atoms to which they are attached denote heterocyclyl,
R′₆ denotes alkyl,
R₇ denotes hydrogen, or
R₆ and R₇ together with the nitrogen atom to which they are attached form heterocyclyl.

In another aspect the present invention provides a pharmaceutically active compound of the present invention which is a compound of formula 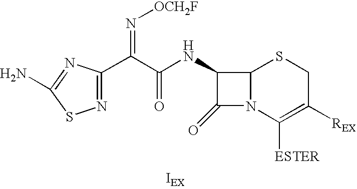
wherein ESTER is as defined above and R_{EX} is a group of formula

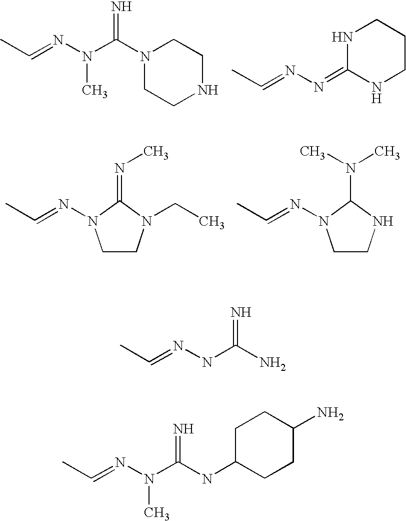

In another aspect the present invention provides a pharmaceutically active compound of the present invention which is a compound of formula 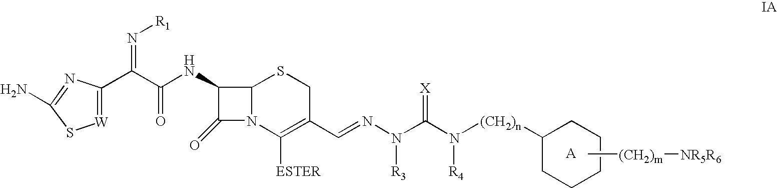
or of formula 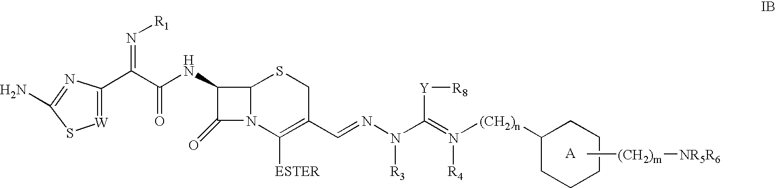
wherein
ESTER is as defined above,
W is CH or N,
R₁ is hydroxy, (C₁₋₆)alkoxy, halo(C₁₋₆)alkoxy, hydroxycarbonyl(C₁₋₆)alkoxy or (C₁₋₆)alkoxycarbonyl(C₁₋₆)alkoxy,
R₃ is hydrogen, (C₁₋₆)alkyl, (C₂₋₆)alkenyl or (C₃₋₈)cycloalkyl,
R₄ is hydrogen or (C₁₋₆)alkyl, 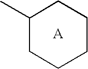
   is cyclohexyl or phenyl,
R₅ and R₆ independently of each other are hydrogen; (C₁₋₆)alkyl; (C₂₋₆)alkenyl; (C₆₋₁₈)arylcarbonyl; (C₁₋₆)alkylcarbonyl; (C₆₋₁₈)aryloxy(C₁₋₄)alkylcarbonyl; (C₁₋₆)alkylcarbonyl-(C₆₋₁₈)arylcarbonyl; heterocyclyl(C₁₋₆)alkylcarbonyl, wherein heterocyclyl comprises 5 or 6 ring members and 1 to 4 heteroatoms selected from N, O or S; (C₁₋₆)alkylsulfonyl or (C₆₋₁₈)arylsulfonyl,
X is NH, O, S or N—R₈, wherein R₈ is (C₁₋₆)alkyl or (C₃₋₈)cycloalkyl,
Y is O or S, and
n and m independently of each other are 0 or 1.

In another aspect the present invention provides a pharmaceutically active compound of the present invention which is a compound of formula 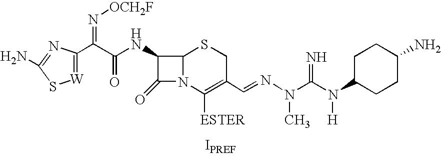
wherein ESTER is as defined above.

In another aspect the present invention provides a pharmaceutically active compound of the present invention which is a cefacetrile, cefaclor, cefadroxil, cefalexin, cefaloglycin, cefaloridine, cefalotin, cefamandole, cefapirin, cefatrizine, cefazedone, cefazolin, cefbuperazone, cefcanel, cefdinir, cefditoren, cefedrolor, cefempidone, cefepime, cefetecol, cefetamet, cefivitril, cefixime, cefluprenam, cefmatilen, cefmenoxime, cefmepidium, cefmetazole, cefminox, cefoperazone, cefodizime, cefpodoxime, cefonicid, cefoperazone, ceforanide, cefoselis, cefotaxime, cefotetan, cefotiam, cefoxitin, cefozopran, cefpimizole, cefluprenam, cefoxazol, cefoxitin, cefpiramide, cefprozil, cefquinome, cefradine, cefpirome, cefroxadine, cefsulodin, ceftazidime, cefteram, ceftezole, ceftibuten, ceftiofur, ceftizoxime, ceftriaxone, cefuroxime, cefuzonam, cephabacin, cephamycin A und B und C, deoxycephamicin B or desacetoxy-cephalosporin C (also as a carbamate), wherein the —COOH group is in the form of a group ESTER, wherein ESTER is as defined above.

In another aspect the present invention provides a pharmaceutically active compound of the present invention which is a compound of formula 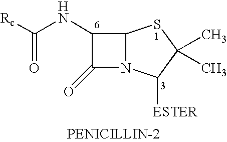
wherein
ESTER is as defined above, and
a) R_{C} is a group of formula 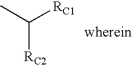
R_{C1} is (C₆₋₁₈)aryl, e.g. phenyl, (C₆₋₁₈)aryloxy, e.g. phenoxy, (C₄₋₈)cyclodialkenyl-heterocyclyl having 5 or 6 ring members and 1 to 4 heteroatoms selected from N, O, S, e.g. thienyl, and
R_{C2} is hydroxyl, (C₁₋₄)alkyl, carboxyl, SO₃H, heterocyclyloxycarbonyl, wherein heterocyclyl has 5 to 6 ring members and 1 to 4 heteroatoms selected from N, O, S, methyleneamino, amino or substituted amino, e.g. amino substituted by heterocyclylcarbonyl, wherein heterocyclyl has 5 or 6 ring members and 1 to 4 heteroatoms selected from N, O, S, amino(C₁₋₄)alkylcarbonyl;
   or
b) R_{c} is heterocyclyl having 5 or 6 ring members and 1 to 4 heteroatoms selected from N, O, S, optionally anellated with another ring (system), or (C₆₋₁₈)aryl, e.g. phenyl, optionally anellated with another ring (system).

“Heterocyclyl” includes heterocyclyl having 5 or 6 ring members and 1 to 4 heteroatoms selected from N, O, S, which may be wholly or partly saturated, e.g. comprising at least one N, which heterocyclyl is optionally anellated with another ring (system), e.g. optionally substuituted heterocyclyl, wherein substituents are selected from hydroxy, (C₁₋₄)alkyl, (C₂₋₆)alkenyl (C₁₋₄)alkoxy, amino(C₁₋₄)alkyl, carboxy(C₁₋₄)alkyl or aminocarbonyl. Heterocyclyl in the meaning of R_{C2} preferably is thienyl and may be unsubstituted or substituted by one or more, especially one or two, substitutents, e.g. substituted by amino.

In another aspect the present invention provides a pharmaceutically active compound of the present invention which is an adicillin, almecillin, amdinocillin, amoxicillin, ampicillin, apalcillin, aspoxicillin, azidocillin, aziocillin, benzylpenicillin, carbenicillin, carindacillin, carfecillin, ciclacillin, clometocillin, cloxacillin, dicloxacillin, epicillin, fenbenicillin, fibracillin, flucloxacillin, fomidacillin, fuzlocillin, hetacillin, metampicillin, methicillin, meziocillin, nafcillin, N-acetylisopenicillin N, oxacillin, penicillin F, penicillin G, penicillin K, penicillin N, penicillin S, penicillin V, penicillin X, pheneticillin, phenoxymethylpenicillin, piperacillin, piroxicillin, propicillin, quinacillin, sulbactam, sulbenicillin, temocillin or ticarcillin, wherein the —COOH group is in the form of a group ESTER, wherein ESTER is as defined above.

In another aspect the present invention provides a pharmaceutically active compound of the present invention which is meropenem or imipenem.

A pharmaceutically active compound provided by the present invention includes e.g. a compound of formula CEPH, PENICILLIN, CARBAPENEM or PENICILLIN-2.

A pharmaceutically active carboxylic acid ester provided by the present invention may be in the form of an physiologically-hydrolysable and -acceptable ester. By physiologically-hydrolysable and -acceptable esters as used herein is meant an ester in which the COO⁻-group is esterified and which is hydrolysable under physiological conditions to yield an acid which is itself physiologically tolerable at dosages to be administered. The term is thus to be understood as defining regular pro-drug forms. An ester moiety may be preferably a group which is easily hydrolysable under physiological conditions. Such esters may be administered preferably orally.

Compounds provided by the present invention, e.g. compounds of formula CEPH, CEPH_{Pref}, PENICILLIN, PENICILLIN-2, CARBAPENEM, I_{PREF}, I_{EX}, IA, IB, I_{EP824535}, I_{EP973780} and I_{WO9948896} are hereinafter designated as “compound(s) of the present invention”. A compound of the present invention includes a compound In any form, e.g. in the form of a salt, in free base form or in the form of a solvate.

In a further aspect the present invention provides a compound of the present invention in the form of a salt, e.g. and/or in the form of a solvate.

Such salts include preferably pharmaceutically acceptable salts, although pharmaceutically unacceptable salts are included, e.g. for preparation/isolation/purification purposes. The present invention thus includes a compound in free base form or, e.g. where such forms exist, in the form of a salt, for example in the form of an acid addition salt, inner salt, quaternary salt, and/or In the form of a solvate, for example In the form of a hydrate. A salt may be a pharmaceutically acceptable salt, such as a metal salt, an amine salt or an acid addition salt. Metal salts include for example sodium, potassium, calcium, barium, zinc, aluminum salts, preferably sodium or potassium salts. Amine salts include salts of a compound of the present invention with an amine, for example trialkylamine, procaine, dibenzylamine and benzylamine salts. Acid addition salts include salts of a compound of formula I with an acid, e.g. hydrogen fumaric acid, fumaric acid, naphthalin-1,5-sulphonic acid, hydrochloric acid, deuterochloric acid. A free form of a compound of the present invention may be converted into a salt/solvate form and vice versa.

A compound of the present invention may exist in the form of isomers and mixtures thereof; e.g. optical isomers, diastereoisomers, cis/trans isomers. A compound of the present invention may e.g. contain asymmetric carbon atoms and may thus exist in the form of enatiomers or diastereoisomers and mixtures thereof, e.g. racemates. Substituents at any asymmetric carbon atom may be present in the (R)-, (S)- or (R,S)-configuration, preferably in the (R)- or (S)-configuration. E.g. the configuration of the OR group in a group —C═N—OR may be syn [(Z)] and anti [(E)] and is preferably syn [(Z)]. E.g. cis/trans isomers may be present, in case that an aliphatic double bond is present in a compound of the present invention. Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of the present invention, where tautomers can exist.

In another aspect the present invention provides a process for the production of a carboxylic acid ester of the present invention comprising the steps

a. reacting a compound of formula R—OH wherein R is as defined above with a compound of formula 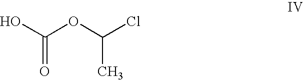
to obtain a compound of formula

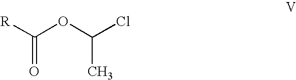

b. reacting a compound of formula V with NaI to obtain a compound of formula 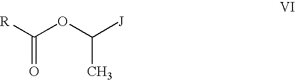
and

c. reacting a compound of formula VI with the —COOH group of a pharmaceutically active compound having a carboxylic acid group —COOH as a part of its chemical structure, and

d. isolating a compound of the present invention obtained from the reaction mixture.

A compound of formula IA or IB may be e.g. obtained by reacting a compound of formula 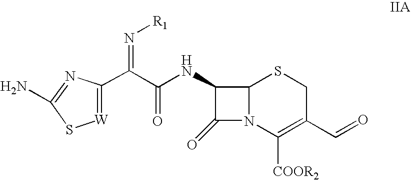
wherein
R₁ and W are as defined above, and
R₂ is a carboxylic group, optionally in in the form of a salt, with a compound of formula 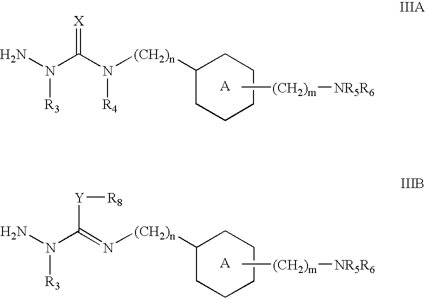
   or
   wherein 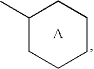
X, Y, R₃, R₄, R₅, R₆, R₈, n and m are as defined above, producing a carboxylic acid ester as described above and isolating a compound of formula IA or IB obtained from the reaction mixture.

In an intermediate of formula IIA or of formula IIIA or IIIB, functional groups, if present, optionally may be in protected form or in the form of a salt, if a salt-forming group is present. Protecting groups, optionally present, may be removed at an appropriate stage, e.g. according, e.g. analogously, to a method as conventional.

If desired, reactive groups in intermediates (starting materials) of the present invention may be protected with protecting groups, which may be or which are split off under the reaction conditions or after termination of the reaction. A compound of the present invention may be isolated from the reaction mixture as appropriate, e.g. according to a method as conventional.

Other pharmaceutically active compound, e.g. cephalosporins, may be prepared according, e.g. anlagously, to the processes as described in WO9635692, WO9843981 or WO9948896 and further esterfying as described herein.

A compound of the present invention, e.g. in free form or in the form of a salt/solvate, exhibits pharmacological activity, e.g. beside low toxicity, and are therefore useful as pharmaceuticals. In particular, the active compounds of the invention show antimicrobial, e.g. antibacterial, activity against e.g. gram negative and gram positive bacteria, e.g. gram positive bacteria such as Escherichia, e.g. Escherichia coli; Enterobacter, e.g. Enterobacter cloacae; Enterococcus, e.g. Enterococcus faecalis; Klebsiella, e.g. Klebsiella pneumoniae; Streptococcus, e.g. Streptococcus pneuminiae; Staphylococcus, e.g. Staphylococcus aureus; and Pseudomonas, e.g. Pseudomonas aeruginosa, in vitro in the Agar Dilution Test according to National Commitee for Clinical Laboratory Standards (NCCLS) 1993, Document M7-A3 Vol. 13, No. 25: “Methods for dilution Antimicrobial Susceptibility Tests for Bacteria that Grow Aerobically—Third Edition, Approved Standard”. The active compounds show a MIC (μg/ml) in the Agar Dilution Test from about <6.4 to about >0.0125. The active compounds of the invention show a surprising overall activity spectrum.

The compounds of the present invention in the form of a salt exhibit the same order of activity as the active compounds of the present invention in free form; optionally in the form of a solvate.

For pharmaceutical use a compound of the present invention includes one or more, preferably one, compounds of the present invention, e.g. a combination of two or more compounds of the present invention.

In another aspect the present invention provides a compound of the present invention for use as a pharmaceutical, preferably as an antimicrobial agent, such as an antibiotic.

In a further aspect the present invention provides a compound of the present invention for use in the preparation of a medicament for the treatment of microbial diseases, for example of diseases caused by bacterias selected from Escherichia, Enterobacter, Enterococcus, Klebsiella, Streptococcus, Staphylococcus and Pseudomonas.

In a further aspect the present invention provides a method of treatment of microbial diseases which comprises administering to a subject in need of such treatment an effective amount of a compound of the present invention.

Treatment includes treatment and prophylaxis.

For such treatment, the appropriate dosage will, of course, vary depending upon, for example, the chemical nature and the pharmakokinetic data of a compound of the present invention employed, the individual host, the mode of administration and the nature and severity of the conditions being treated. However, in general, for satisfactory results in larger mammals, for example humans, an indicated daily dosage is in the range from about 0.05 to about 5 g (e.g. from about 0.625 mg/kg to about 62.5 mg/kg), for example from about 0.1 to about 2.5 g (e.g. from about 1.25 mg/kg to about 31.25 mg/kg), of an active compound of the invention conveniently administered, for example, in divided doses up to four times a day.

A compound of the present Invention may be administered by any conventional route, for example enterally, e.g. including nasal, buccal, rectal, oral, administration; parenterally, e.g. including intravenous, intramuscular, subcutanous administration; or topically; e.g. including epicutaneous, intranasal, intratracheal administration; e.g. in form of coated or uncoated tablets, capsules, (injectable) solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories, preferably orally. e.g. in form of coated or uncoated tablets, capsules, solid solutions, suspensions, dispersions, solid dispersions, powders.

Because of activity against various e.g. bacterial strains, compounds of the present invention are indicated for the treatment of microbial diseases, e.g. bacterial diseases. The compounds of the invention may be administered to larger mammals, for example humans, by similar modes of administration at similar dosages than conventionally employed with cefuroxim axetil. The compound of the present invention may be administered in pharmaceutically acceptable salt form, e.g. acid addition salt form or base addition salt form or in the corresponding free forms, optionally in solvate form. Such salts exhibit the same order of activity as the free forms.

The present invention also provides a pharmaceutical composition comprising a compound of the present invention in association with at least one pharmaceutical excipient, e.g. carrier or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers, e.g. further comprising another pharmaceutically active agent

Such compositions may be manufactured accordingly, e.g. analogously to a method as conventional.

Other pharmaceutical agents include e.g. other antibiotics, preferably such which may be administered orally.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

In a further aspect the present invention provides the use of an ester group selected from the group consisting of
1-(2,3-disubstituted 1-propoxycarbonyloxy)-ethyl carboxylic acid ester, wherein the substituents are selected from the group consisting of hydroxy and (C₁₋₂₂)alkylcarbonyloxy,
1-(1,3-disubstituted 2-propoxycarbonyloxy)-ethyl carboxylic acid ester, wherein the substituents are selected from the group consisting of hydroxy and (C₁₋₂₂)alkylcarbonyloxy,
1-(9H-fluorene-9-yl-(C-₁₋₄)alkanyloxycarbonyloxy)-ethyl carboxylic acid ester, and
1-(decahydro-naphthalene-2-yl-oxycarbonyloxy)-ethyl carboxylic acid ester for the improvement of oral resorption of a pharmaceutically active compound.

In a further aspect the present invention provides the use of an ester group selected from the group consisting of
1-(2,3-disubstituted 1-propoxycarbonyloxy)-ethyl carboxylic acid ester, wherein the substituents are selected from the group consisting of hydroxy and (C₁₋₂₂)alkylcarbonyloxy,
1-(1,3-disubstituted 2-propoxycarbonyloxy)-ethyl carboxylic acid ester, wherein the substituents are selected from the group consisting of hydroxy and (C₁₋₂₂)alkylcarbonyloxy,
1-(9H-fluorene-9-yl-(C₁₋₄)alkanyloxycarbonyloxy)-ethyl carboxylic acid ester,
1-(decahydro-naphthalene-2-yl-oxycarbonyloxy)-ethyl carboxylic acid ester and
1-(2-amino(C₁₋₄)alkoxy-carbonyloxy)-ethyl carboxylic acid ester,
   for the improvement of oral resorption of a pharmaceutically active compound, with the proviso that, if the pharmaceutically active compound is a penicillin, then 1-(2-amino-(C₁₋₄)alkoxycarbonyloxy)-ethyl carboxylic acid esters are excluded.

In another aspect the present invention provides a carboxylic acid ester of a pharmaceutically active compound having a carboxylic acid group —COOH as a part of its chemical structure, which ester is selected from the group consisting of 1-(1,3-disubstituted propoxycarbonyloxy)-ethyl carboxylic acid ester, 1-(2,3)-disubstituted propoxycarbonyloxy)-ethyl carboxylic acid ester, 1-(9H-fluorene-9-yl-(C₁₋₄)alkanyloxycarbonyloxy)-ethyl carboxylic acid ester, 1-(decahydro-naphthalene-2-yl-oxycarbonyloxy)-ethyl carboxylic acid ester and 1-(2-amino(C₁₋₆)alkoxycarbonyloxy)-ethyl carboxylic acid ester, with the PROVISO that if the pharmaceutically active compound is a penicillin, then a 1-(2-amino(C₁₋₄)alkoxycarbonyloxy)-ethyl carboxylic acid ester is excluded.

In the following examples all temperatures are given in degree centigrade and are uncorrected. RT means room temperature. EX. is Example.

### EXAMPLES

### Example 1

A) Carbonic acid 1-chloro-ethyl ester 2,2-dimethyl-[1,3]dioxolan-4-ylmethyl ester

20.3 g of 4-methylmorpholine, 230 mg of 4-dimethylaminopyridine and 26.1 g of chloroformic acid 1-chloro-ethyl ester are added at 40 to 24 g of (2,2-dimethyl-[1,3]dioxolan-4-yl)-methanol in 450 ml of CH₂Cl₂. The mixture obtained is stirred, a salt precipitated is filtered off and the mixture obtained is subjected to chromatography. Carbonic acid 1-chloro-ethyl ester 2,2-dimethyl-[1,3]dioxolan-4-yl-methyl ester is obtained.

B) Carbonic acid 2,2-dimethyl-[1,3]dioxolan-4-ylmethyl ester 1-iodo-ethyl ester

40 g of carbonic acid 1-chloro-ethyl ester 2,2-dimethyl-[1,3]dioxolan-4-ylmethyl ester in 300 ml of acetonitrile are added to 151.2 g of NaI in 1.25 l of acetonitrile. The mixture obtained is stirred, a salt precipitated is filtered off and dissolved in ether. The mixture obtained is subjected to extractive washing and solvent is evaporated from the organic layer obtained. Carbonic acid 2,2-dimethyl-[1,3]dioxolan-4-ylmethyl ester 1-iodo-ethyl ester is obtained.

C) 7-{2-(5-Amino-[1,2,4]thiadiazol-3-yl)-2-(fluoromethoxyimino)-acetylamino}-3-formyl-8-oxo-5-thia-1-aza-bicyclo[4.2.0]oct-2-ene-2-carboxylic acid 1-(2,2-dimethyl-[1,3]dioxolan-4-yl-methoxycarbonyloxy)-ethyl ester

38 g of carbonic acid 2,2-dimethyl-[1,3]dioxolan-4-ylmethyl ester 1-iodo-ethyl ester in 200 ml of dimethylacetamide are added to 41.5 g of 7-{2-(5-Amino-[1,2,4]thiadiazol-3-yl)-2-[(Z)-fluoromethoxyimino]-acetylamino}-3-formyl-8-oxo-5-thia-1-aza-bicyclo[4.2.0]oct-2-ene-2-carboxylic acid sodium salt in 840 ml of dimethylacetamide. A reaction mixture formed is stirred, poured into 2 l of an ice-H₂O mixture and the mixture obtained is extracted with ethylacetate. The organic layer obtained is washed with saturated Na₂CO₃-solution, brine, dried, concentrated and a residue formed is triturated with ether. 7-{2-(5-Amino-[1,2,4]thiadiazol-3-yl)-2-(fluoromethoxyimino)-acetylamino}-3-formyl-8-oxo-5-thia-1-aza-bicyclo[4.2.0]oct-2-ene-2-carboxylic acid 1-(2,2-dimethyl-[1,3]dioxolan-4-yl-methoxycarbonyloxy)-ethyl ester is obtained.

¹H-NMR (DMSO-d₆): 1.22 (s, 3H); 1.30 (2xs, 3H); 1.56 (d, 3H, J=6 Hz); 3.45 (d, 1H, J=18 Hz); 3.64-4.30 (m, 6H); 5.33 (2xd, 1H, J=5 Hz); 5.68 (m, 1H); 5.83 (m, 1H), 6.05 (2xdd, 1H, J=5 Hz, 8 Hz); 6.90/6.97 (2xq, 1H, J=6 Hz); 8.20 (s, 1H); 9.62 (d, 1H, J=6 Hz); 9.84 (d, 1H, J=8 Hz)

D) 7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester

12.2 g of N-amino N-methyl-piperazine-1-carboxamidine, 19.4 ml of 2N HCl and 45 ml of H₂O are added to 24.5 g of 7-{2-(5-Amino-[1,2,4]thiadiazol-3-yl)-2-[(Z)-fluoromethoxyimino]-acetylamino}-3-formyl-8-oxo-5-thia-1-aza-bicyclo[4.2.0]oct-2-ene-2-carboxylic acid 1-(2,2-dimethyl-[1,3]dioxolan-4-ylmethoxycarbonyloxy)-ethyl ester in 950 ml of dimethylacetamide. A reaction mixture formed is allowed to stand at ambient temperature and from the mixture obtained, solvent is evaporated. The evaporation residue obtained is triturated with ether and the mixture obtained is cooled. 150 ml of HCl-saturated ether are added, the mixture formed is stirred and ether is decanted. The decantation residue obtained is washed, dried and optionally subjected to chromatography. 7-{2-(5-Amino-[1,2,4]thiadiazol-3-yl)-2-(fluoro-methoxyimino)-acetylamino}-3-formyl-8-oxo-5-thia-1-aza-bicyclo[4.2.0]oct-2-ene-2-carboxylic acid 1-(2,2-dimethyl-[1,3]dioxolan-4-ylmethoxycarbonyloxy)-ethyl ester is obtained.

Analogously to procedures as described in example 1, but using appropriate starting materials, compounds of formula

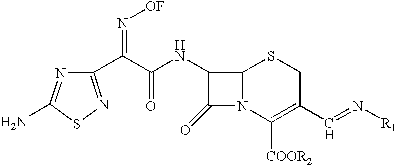

wherein R1 and R2 are as defined in TABLE 1 below are obtained. Purification may be carried out optionally.

TABLE 1

| | | | |
|---|---|---|---|
| Ex. | R¹ | R² | ¹H-NMR (d₆-DMSO) |

| | | | |
|---|---|---|---|
| 1 | 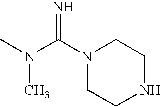 | 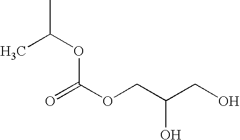 | 1.53 (d, 3 H, J=6 Hz); 3.21 (bs, 1 H); 3.27-3.38 (m, 4 H); 3.61 (2 xd, 1 H, J=18 Hz); 3.64-3.74 (m, 7 H); 3.99 (m, 1 H); 4.18 (m, 1 H); 4.31/4.35 (2 xd, 1 H, J=18 Hz); 5.29-5.34 (2 xd, 1 H, J=5 Hz); 5.72 (m, 1 H); 5.83 (m, 1 H,); |
| # 5.94-6.01 (2 xq, 1 H, J=5 Hz, 8 Hz); 6.84-6.94 (2 xq, 1 H, J=5 Hz); 7.91/7.94 (2 xs, 1 H); 8.23 (s, 2 H); 9.01 (bs, 1 H); 9.25 (bs, 1 H); 9.62 (bs, 2 H); 9.82 (2 xd, 1 H, J=8 Hz) | | | |
| 2 | 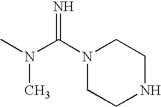 | 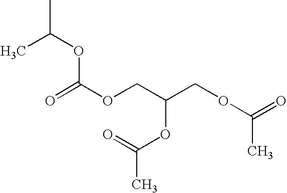 | 1.56 (d, 3 H, J=6 Hz); 2.06 (2 xs, 6 H); 3.26 (bs, 3 H); 3.37 (bs, 4 H); 3.62-3.65 (m, 1 H); 3.73 (m, 4 H); 4.13-4.39 (m, 5 H); 5.20 (m, 1 H); 5.33-5.35 (m, 1 H,); 5.72 (bs, 1 H); 5.85 (bs, 1 H), 5.99 (m, 1 H,); 6.89-6.98 (m, 1 H,); 8.02 (2 xs, 1H); 9.03 (bs, 1 H); 9.25 (bs, 1 H); 9.67 (bs, 2 H,); 9.85 (d, 1 H, J=8 Hz) |
| 3 | 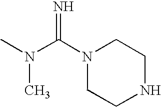 | 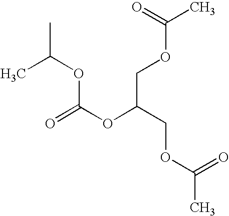 | 1.58 (d, 3 H, J=6 Hz); 2.03 (2 xs, 6 H); 3.28 (m, 4 H); 3.30 (bs, 3 H); 3.63 (d, 1 H, J=18 Hz); 3.72 (m, 4 H); 4.13-4.33 (m, 4 H); 4.39 (d, 1 H, J=18 Hz); 5.06-5.22 (m, 1 H); 5.32 (d, 1 H, J=5 Hz); 5.73 (m, 1 H); 5.86 (m, 1 H), 6.00 (dd, 1 H, J=6 Hz, 9 Hz); 6.98 |
| # (m, 1 H,); 8.05 (s, 1 H); 8.27 (bs, 2 H); 9.04 (bs, 1 H); 9.28 (bs, 1 H); 9.64 (bs, 2 H,); 9.84(m, 1 H) | | | |
| 4 | 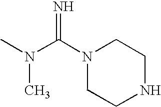 | 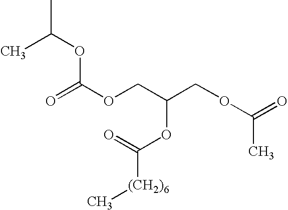 | 0.87 (t, 3 H, J=6 Hz); 1.25 (m, 8 H); 1.50 (m, 2 H); 1.56 (d, 3 H, J=6 Hz); 2.03 (2 xs, 3 H); 2.30 (m, 2 H); 3.25 (m, 4 H); 3.30 (m, 3 H); 3.58-3.62 (m, 1 H,); 3.72 (m, 4 H); 4.16-4.35 (m, 4 H); 4.38-4.41 (m, 1 H,); 5.05-5.20 (m, 1 H); 5.30-5.35 (2 xd, 1 H, J=6 Hz); |
| # 5.73 (m, 1 H); 5.83 (m, 1 H), 6.00 (m, 1 H,); 6.90-6.98 (2 xq, 1 H, J=6 Hz); 7.98-8.03 (2 xs, 1 H); 8.27 (m, 2 H); 9.03 (bs, 1 H); 9.30 (bs, 1 H); 9.67 (bs, 2 H,); 9.87 (m, 1 H) | | | |
| 5 | 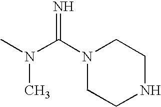 | 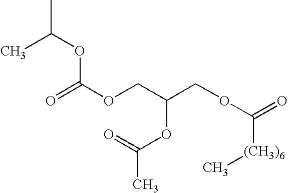 | 0.87 (t, 3 H, J=7 Hz); 1.25 (m, 8 H); 1.52 (m, 2 H); 1.57 (d, 3 H, J=6 Hz); 2.22 (2 xs, 3 H); 2.30 (t, 2 H, J=7 Hz); 3.27 (m, 4 H); 3.33 (bs, 3 H); 3.63 (m, 1 H,); 3.73 (m, 4 H); 4.12-4.41 (m, 5 H); 5.20 (m, 1 H); 5.33 (2 xd, 1 H, J=6 Hz); 5.73 (bs, 1 H); 5.85 (bs, 1 H), |
| # 5.97-6.03 (m, 1 H,); 6.88-6.99 (m, 1 H); 7.98-8.06 (m, 1 H); 8.28 (bs, 2 H); 9.06 (bs, 1 H); 9.30 (bs, 1 H); 9.67 (bs, 2 H,); 9.85 (t, 1 H J=8 Hz) | | | |
| 6 | 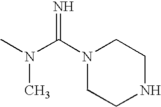 | 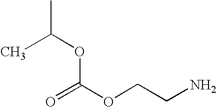 | 1.56 (d, 3 H, J=6 Hz); 3.10 (m, 2 H); 3.20 (m, 4 H); 3.30 (2 xs, 3 H); 3.60 (m, 1 H); 3.72 (m, 4 H); 4.32 (m, 3 H); 5.32 (2 xd, 1 H, J=5 Hz); 5.70 (bs, 1 H); 5.83 (bs, 1 H), 5.97 (m, 1 H); 6.90-6.98 (2 xq, 1 H, J=6 Hz); 7.96 (2 xs, 1 H); 8.25 (bs, 2 H); 8.30 (bs, 2 H); |
| # 9.10 (bs, 1 H); 9.30 (bs, 1 H); 9.70 (bs, 2 H,); 9.73 (m, 1 H) | | | |
| 7 | 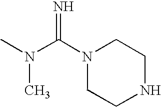 | 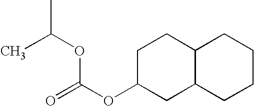 | 1.25-1.85 (m, 16 H); 1.56 (d, 3 H, J=6 Hz); 3.15 (m, 4 H); 3.27 (2 xs, 3 H); 3.60 (m, 1 H); 3.62 (m, 4 H); 1 H); 5.19/5.36 (2 xd, 1 H, J=6 Hz); 5.73-5.87 (m, 2 H); 5.99 (m, 1 H,); 6.60-6.98 (2 xm, 1 H); 7.40/7.72 (2 xm, 1 H); 8.25 (m, 3 H); 9.85-9.92(m, 1 H) |
| 8 | 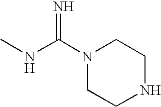 | 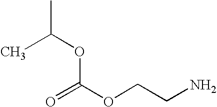 | 1.56 (2 xd, 3 H, J=6 Hz); 3.10-3.90 (m, 8 H); 3.20 (m, 2 H); 3.53 (m, 1 H); 4.33 (m, 2 H); 4.63 (m, 1 H); 5.31 (2 xd, 1 H, J=5 Hz); 5.70 (m, 1 H); 5.82 (m, 1 H), 5.93-6.02 (2 xdd, 1 H, J=5 Hz, 8 Hz); 6.72-6.97 (2 xq, 1 H, J=6 Hz); 8.25 (m, 2 H); 8.60-8.70 (2 xs, 1 H); 9.75 (bs, 2H); 9.80 (2 xd, 1 H, J=8 Hz) |
| 9 | 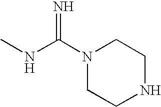 | 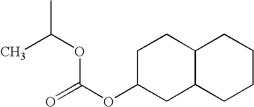 | 1.20-1.87 (m, 16 H); 1.56 (d, 3 H, J=6 Hz); 3.15-3.47(m, 8 H); 3.55 (d, 1 H, J=18 Hz); 4.75 (m, 1 H); 5.30 (2 xd, 1 H, J=5 Hz); 5.72 (m, 1 H); 5.83 (m, 1 H); 6.02 (2 xm, 1 H,); 6.85/6.95 (2 xm, 1 H); 8.52/8.62 (2 xs, 1 H); 9.70 (m, 1 H) |
| 10 | 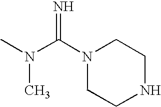 | 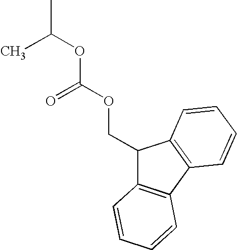 | 1.49 (d, 3 H, J=5 Hz); 3.21 (bs, 3 H); 3.30-3.70 (m, 8 H); 3.60 (d, 1 H, J=18 Hz); 4.30 (m, 1 H); 4.31 (d, 1 H, J=18 Hz); 4.60 (m, 2 H); 5.30 (2 xd, 1 H, J=5 Hz); 5.69 (m, 1 H); 5.83 (m, 1 H,); 5.92-6.02 (2 xm, 1 H); 6.82/6.91 (2 xq, 1 H, J=5 Hz); 7.33 (m, 2 H); 7.42 (m, 2 H); 7.62 (m, 2 H); 7.88 (m, 2 H); 7.93 (s, 1 H); 8.22 (bs, |
| # 1 H); 8.98 (bs, 1 H); 9.22 (bs, 1 H); 9.63 (bs, 1 H); 9.83 (2 xd, 1 H, J=8 Hz) | | | |
| 11 | 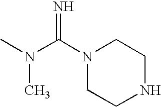 | 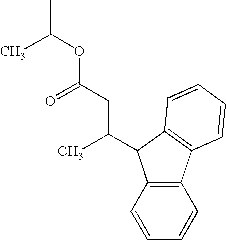 | 0.72-0.84 (3 xd, 3 H, J=6 Hz); 1.45-1.63 (3 xd, 3 H, J=6 Hz); 3.30-3.70 (m, 8 H); 3.33 (4 xs, 3 H); 3.62 (d, 1 H, J=18 Hz); 4.33 (d, 1 H, J=18 Hz); 4.43 (m, 1 H); 5.20-5.99 (m, 5 H); 6.65-7.00 (3 xq, 1 H, J=6 Hz); 7.30-8.30 (m, 9 H); 9.03-9.30 (m, 3 H); 9.83-9.93 (m, 2 H) |
| 12 | 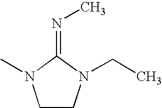 | 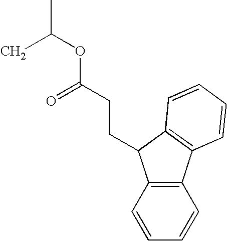 | 1.20 (2 xt, 3 H, J=7 Hz); 1.51 (d, 3 H, J=5 Hz); 3.19 (s, 3 H); 3.53-3.83 (m, 7 H); 4.29-4.40 (m, 2 H); 4.53-4.68 (m, 2 H); 5.33 (2 xd, 1 H, J=5 Hz); 5.72 (m, 1 H); 5.83 (m, 1 H,); 5.93/6.01 (2 xdd, 1 H, J=5 Hz, 8 Hz); 6.83/6.91 (2 xq, 1 H, J=5 Hz); 7.31-7.39 (m, 2 H); 7.40-7.46 (m, 2 H); 7.62-7.68 (m, 3 H); 7.88-7.92 (m, |
| # 2 H); 8.23 (bs, 1 H); 8.62 (bs, 1 H); 9.85 (2 xd, 1 H, J=8 Hz) | | | |
| 13 | 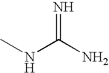 | 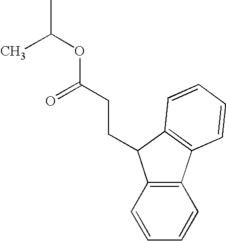 | 1.50 (d, 3 H, J=6 Hz); 3.55 (d, 1 H, J=18 Hz); 4.33 (t, 1 H, J=6 Hz); 4.57 (d, 1 H, J=18 Hz); 4.64 (m, 2 H); 5.31 (2 xd, 1 H, J=5 Hz); 5.71 (m, 1 H); 5.82 (m, 1 H,); 5.93/6.02 (2 xdd, 1 H, J=5 Hz, 8 Hz); 6.71/6.91 (2 xq, 1 H, J=6 Hz); 7.31-7.48 (m, 4 H); 7.65 (m, 2 H); 7.80 (bs, 2 H); 7.95 (m, 2 H); |
| # 8.25-8.30 (2 xs, 1 H); 8.27 (bs, 2 H); 9.85 (2 xd, 1 H, J=8 Hz); 12.10 (bs, 1 H) | | | |
| 14 | 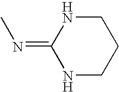 | 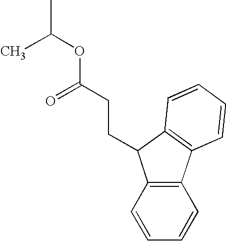 | 1.52 (d, 3 H, J=6 Hz); 1.90 (m, 2 H); 3.35 (m, 4 H); 3.55 (d, 1 H, J=18 Hz); 4.32 (t, 1 H, J=6 Hz); 4.59 (2 xd, 1 H, J=18 Hz); 4.62 (m, 2 H); 5.31 (2 xd, 1 H, J=5 Hz); 5.71 (m, 1 H); 5.83 (m, 1 H,); 5.91/6.01 (2 xdd, 1 H, J=5 Hz, 8 Hz); 6.80/6.90 (2 xq, 1 H, J=6 Hz); 7.32-7.43 (m, 4 H); 7.63 (m, |
| # 2 H); 7.90 (m, 2 H); 8.35 (s, 1 H); 8.45 (s, 1 H); 9.85 (2 xd, 1 H, J=8 Hz) | | | |
| 15 | 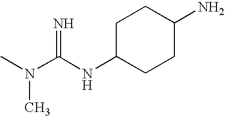 | 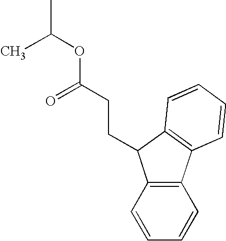 | 1.50-1.59 (m, 4 H); 1.52 (d, 3 H, J=6 Hz); 1.92-2.03 (m, 4 H); 2.94 (m, 1 H); 3.27 (2 xs, 3 H); 3.57 (d, 1 H, J=18 Hz); 3.63 (m, 1 H); 4.32 (m 1 H); 4.53-4.68 (m, 3 H); 5.32 (2 xd, 1 H, J=5 Hz); 5.72 (m, 1 H); 5.83 (m, 1 H,); 5.95/6.01 (2 xdd, 1 H, J=5 Hz, 8 Hz); 6.84/6.91 (2 xq, 1 H, J=6 Hz); 7.31-7.38 |
| # (m, 2 H); 7.42-7.47 (m, 2 H); 7.63 (m, 2 H); 7.89 (m, 2 H); 7.91 (2 xs, 1 H); 8.05 (bs, 1 H); 8.20 (bs, 2 H); 8.27 (s, 2 H); 8.48 (bs, 1 H); 9.85 (2 xd, 1 H, J=8 Hz) | | | |
| 16 | 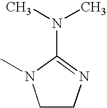 | 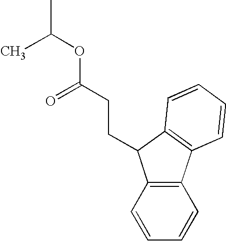 | 1.51 (2 xd, 3 H, J=6 Hz); 3.23 (bs, 6 H); 3.59 (m, 2 H); 3.68 (m, 1 H,); 3.88 (m, 2 H); 4.05 (m, 1 H; J=18 Hz); 4.34 (m, 1 H); 4.62 (m, 2 H); 5.32 (2 xd, 1 H, J=5 Hz); 5.73 (m, 1 H); 5.85 (m, 12 H); 5.96/6.02 (2 xdd, 1 H, J=5 Hz, 8 Hz); 6.84/6.92 (2 xq, 1 H J=6 Hz); 7.37 (m, 2 H); 7.42 (m, 2 H); 7.65 (m, 2 H); 7.90 (m, 2 H); |
| # 8.24 (bs, 1 H); 9.30 (bs, 1 H); 9.87 (2 xd, 1 H, J=8 Hz) | | | |
| 17 | 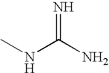 | 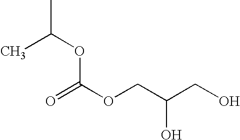 | 1.56 (d, 3 H, J=6 Hz); 3.38 (m, 1 H); 3.57 (d, 1 H, J=18 Hz); 3.68 (m, 2 H); 3.99 (ABX-system, 1 H, J_{AB} = 10 Hz, J_{AX} = 6 Hz); 4.22 (ABX- system, 1 H, J_{AB} = 10 Hz, J_{BX} = 5 Hz); 4.55 (d, 1 H, J=18 Hz); 5.31 (2 xd, 1 H, J=5 Hz); 5.73 (m, 1 H); 5.83 (m, 1 H,); 5.97 (dd, 1 H, J=5 Hz, 8 Hz); 6.95 (q, 1 H, |
| # J=5 Hz); 8.21 (s, 2 H); 8.30 (s, 1 H); 9.82 (d, 1 H, J=8 Hz); 12.05 (s, 1 H) | | | |
| 18 | 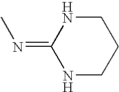 | 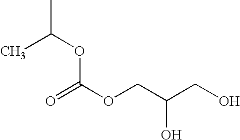 | 1.56 (2 xd, 3 H, J=6 Hz); 1.90 (m, 2 H); 3.29-3.39 (m, 5 H); 3.55 (d, 1 H, J=18 Hz); 3.69 (m, 2 H); 4.00 (m, 1 H); 4.20 (m, 1 H); 4.57 (2 xd, 1 H, J=18 Hz); 4.75 (m, 1 H); 5.08 (m, 1 H); 5.33 (2 xd, 1 H, J=5 Hz); 5.71 (m, 1 H); 5.85 (m, 1 H,); 5.95/6.02 (2 xdd, 1 H, J=5 Hz, 8 Hz); 6.85/6.92 (2 xq, 1 H, |
| # J=5 Hz); 8.25 (s, 1 H); 8.42 (s, 1 H); 9.85 (2 xd, 1 H, J=8 Hz); 12.06 (bs, 1 H) | | | |
| 19 | 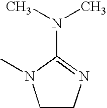 | 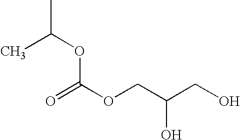 | 1.57 (2 xd, 3 H, J=6 Hz); 3.20 (2 xs, 3 H); 3.28-3.40 (m, 7 H); 3.66 (m, 1 H,); 3.72 (m, 2 H); 3.93 (m, 1 H); 4.02-4.08 (m, 2 H); 4.20 (m, 1 H); 5.31 (2 xd, 1 H, J=5 Hz); 5.70-5.89 (m, 2 H); 5.95/6.03 (m, 1 H); 6.81/6.98 (m, 1 H); 7.73 (m, 1 H); 8.12-8.22 (m, 4 H); 9.82 (2 xd, 1 H, J=8 Hz |
| 20 | 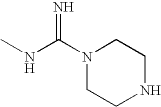 | 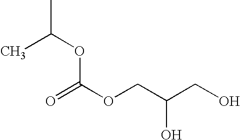 | 1.50 (2 xd, 3 H, J=6 Hz); 3.20-3.80 (m, 12 H); 3.93 (m, 1 H); 4.13 (m, 1 H); 4.57/4.62 (2 xd, 1 H, J=18 Hz); 5.23-5.34 (2 xd, 1 H, J=5 Hz); 5.66 (m, 1 H); 5.80 (m, 1 H,); 5.90-5.98 (2 xq, 1 H, J=5 Hz, 8 Hz); 6.78/6.84 (2 xq, 1 H, J=5 Hz); 8.20 (s, 2 H); 8.48/8.59 (2 xs, 1 H); 8.50 (bs, 1 H); 9.58 (bs, 2 H); |
| # 9.78 (2 xd, 1 H, J=8 Hz); 12.20/12.31 (2 xbs, 1 H) | | | |
| 21 | 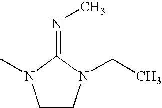 | 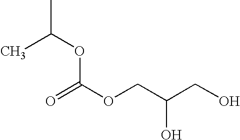 | 1.21 (2 xt, 3 H, J=7 Hz); 1.56 (2 xd, 3 H, J=7 Hz); 3.18 (bs, 3 H); 3.30-3.42 (m, 2 H); 3.57-3.71 (m, 4 H); 3.80-3.88 (m, 4 H); 3.94 (m, 1 H); 3.98-4.07 (m, 1 H); 4.17-4.22 (m, 1 H); 4.76 (m, 1 H); 5.07 (t, 1 H, J=5 Hz); 5.36 (2 xd, 1 H, J=5 Hz); 5.74 (m, 1 H); 5.86 (m, 1 H,); 6.01 (m, 1 H); 6.88/6.96 |
| # (2 xq, 1 H, J=5 Hz); 7.71 (2 xs, 1 H); 8.25 (bs, 2 H); 8.58 (bs, 1 H); 9.85 (2 xd, 1 H, J=8 Hz) | | | |
| 22 | 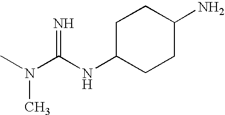 | 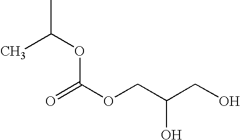 | 1.50-1.59 (m, 4 H); 1.52 (d, 3 H, J=6 Hz); 1.92-2.03 (m, 4 H); 2.92 (m, 1 H); 3.25 (m, 1 H); 3.27 (2 xs, 3 H); 3.50 (m, 1 H); 3.57 (d, 1 H, J=18 Hz); 3.63 (m, 2 H); 3.96 (m, 1 H); 4.21 (m, 1 H); 4.62 (d, 1 H, J=18 Hz); 5.27 (2 xd, 1 H, J=5 Hz); 5.72 (m, 1 H); 5.83 (m, 1 H,); 5.95/6.01 (2 xdd, 1 H, J=5 Hz, 8 Hz); 6.84/6.91 |
| # (2 xq, 1 H, J=6 Hz); 7.91 (s, 1 H); 8.05 (bs, 1 H); 8.20 (bs, 2 H); 8.27 (s, 2 H); 8.48 (bs, 1 H); 9.85 (2 xd, 1 H, J=8 Hz) | | | |

### Example 23

3-Carbamoyloxymethyl-7-{2-furan-2-yl-2-[(Z)-methoxyimino]-acetlamino}-8-oxo-5-thia-1-aza-bicyclo[4.2.0]oct-2-ene-2-carboxylic acid 1-(9H-fluoren-9-yl-methoxycarbonyloxy)-ethyl ester

38 g of carbonic acid 2,2-dimethyl-[1,3]dioxolan-4-ylmethyl ester 1-iodo-ethyl ester in 200 ml of dimethylacetamide are added to 41.5 g of cefuroxim in the form of a sodium salt in 840 ml of dimethylacetamide. The mixture obtained is stirred, poured into 2 l of an ice-H₂O mixture and the mixture obtained is extracted with ethyl acetate. The organic layer obtained is washed with saturated Na₂CO₃-solution, brine, dried, concentrated and the concentration residue obtained triturated with ether. 3-Carbamoyloxymethyl-7-{2-furan-2-yl-2-[(Z)-methoxyimino]-acetylamino}-8-oxo-5-thia-1-aza-bicyclo[4.2.0]oct-2-ene-2-carboxylic acid 1-(9H-fluoren-9-yl-methoxycarbonyloxy)-ethyl ester is obtained.

¹H-NMR (DMSO-d₆): 1.48 (d, 3H, J=6 Hz); 3.55 (d, 1H, J=18 Hz); 3.64 (2xd, 1H, J=18 Hz); 3.89 (2xs, 3H); 4.31-4.38 (m, 1H); 4.49-4.86 (m, 4H); 5.22 (2xd, 1H, J=5 Hz); 5.58-5.89 (m, 1H); 6.60-6.73 (m, 2H); 6.78/6.89 (2xq, 1H, J=6 Hz); 7.32-7.38 (m, 2H); 7.42-7.46 (m, 2H); 7.62-7.68 (m, 2H); 7.83 (m, 1H); 7.92 (m, 2H); 9.79 (2xd, 1H, J=8 Hz)

Analogously to example 23 but using the appropriate starting materials the following compound is prepared.

### Example 24

3Carbamoyloxymethyl-7-{2-furan-2-yl-2-[(Z)-methoxyimino]-acetylamino}-8-oxo-5-thia-1-aza-bicyclo[4.2.0]oct-2-ene-2-carboxylic acid 1-(2,3-dihydroxy-propoxycarbonyloxy)-ethyl ester

¹H-NMR (DMSO-d₆): 1.50 (d, 3H, J=6 Hz); 3.30 (m, 2H); 3.49/3.62 (2xd, 1H, J=18 Hz); 3.63 (m, 1H) 4.00 (s, 3H); 4.03 (m, 1H); 4.18 (m, 1H); 4.75/4.80 (m, 1H); 5.18-5.24 (2xd, 1H, J=5 Hz); 5.80-5.87 (m, 1H); 6.67 (m, 1H); 6.78/6.87 (2xq, 1H, J=6 Hz); 7.22 (d, 1H, J=3.5 Hz); 7.83 (m, 1H); 9.57 (2xd, 1H, J=8 Hz)

### INTERMEDIATES

Intermediates of formulae

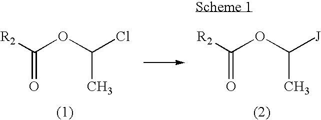

wherein R₂ is as described in TABLE 1 above, are obtained analogously as described in Example 1, but using appropriate starting materials. The ¹H-NMR data of such intermediates is set out in TABLE 2 below.

TABLE 2

| | Chloride(1) | Iodide(2) |
|---|---|---|
| Ex′ | ¹H-NMR(CDCl₃) | ¹H-NMR(CDCl₃) |
| 1′ | 1.36(s, 3H); 1.43(2xs, 3H); 1.83(d, 3H, J=6Hz); | ¹H-NMR(CDCl₃): 1.36(s, 3H); 1.44(2xs, 3H); 2.25(2xd, 3H, |
| | 3.79(m, 1H); 4.09(m, 1H); 4.23(m, 2H); 4.34(m, 1H); | J=6Hz); 3.78(m, 1H); 4.09(m, 1H); 4.22(m, 2H); |
| | 6.42(q, 1H, J=6Hz) | 4.33(m, 1H); 6.75(m, 1H, ) |
| 2′ | 1.86(d, 3H, J=6Hz); 2.11(s, 3H); 2.20(s, 3H); | 2.08(s, 3H); 2.12(s, 3H); 2.25(d, 3H, J=6Hz); |
| | 4.20(m, 1H); 4.34(m, 2H); 4.44(m, 1H); 5.28(m, 1H); | 4.18(m, 1H); 4.30(m, 2H); 4.42(m, 1H); 5.24(m, 1H); |
| | 6.43(2xq, 1H, J=6Hz) | 6.74(2xq, 1H, J=6Hz) |
| 3′ | 1.84/1.86(2xd, 3H, J=6Hz); 2.08-2.10(4xs, 6H); | 2.10(2xs, 3H); 2.27(d, 3H, J=6Hz); 4.18(m, 2H); |
| | 4.17-4.40(m, 2H); 5.14-5.28(m, 1H); | 4.35(m, 2H); 5.18-5.28(m, 1H); 6.74(2xq, 1H, J=6Hz) |
| | 6.41-6.42(2xq, 1H, J=6Hz) | |
| 4′ | 0.90(t, 3H, J=6Hz); 1.30(m, 8H); 1.63(m, 2H); | 0.90(t, 3H, J=6Hz); 1.32(m, 8H); 1.64(m, 2H); |
| | 1.87(d, 3H, J=6Hz); 2.10(s, 3H); 2.34(t, 2H, J=7Hz); | 2.10(s, 3H); 2.28(d, 3H, J=6Hz); 2.36(m, 2H); |
| | 4.20(m, 2H); 4.38(m, 2H); 5.20(m, 1H); 6.44(q, 1H, J=6Hz) | 4.20(m, 2H); 4.38(m, 2H); 5.20(m, 1H); 6.78(q, 1H, J=6Hz) |
| 5′ | 0.87(t, 3H, J=6Hz); 1.28(m, 8H); 1.62(m, 2H); | 0.87(t, 3H, J=6Hz); 1.28(m, 8H); 1.62(m, 2H); |
| | 1.84(d, 3H, J=6Hz); 2.10(s, 3H); 2.32(m, 2H); | 2.10(s, 3H); 2.24(d, 3H, J=6Hz); 2.32(m, 2H); |
| | 4.30(m, 2H); 4.42(m, 2H); 5.23(m, 1H); 6.40(q, 1H, J=6Hz) | 4.18(m, 2H); 4.30(m, 2H); 5.23(m, 1H); 6.74(q, 1H, J=6Hz) |
| 6′ | 1.44(s, 9H); 1.83(d, 3H, J=6Hz); 3.44(m, 2H); | 1.44(s, 9H); 2.24(d, 3H, J=6Hz); 3.44(m, 2H); |
| | 4.27(t, 2H, J=5Hz); ); 6.42(q, 1H, J=6Hz) | 4.27(t, 2H, J=5Hz); 6.76(q, 1H, J=6Hz) |
| 7′ | 0.90-1.97(m, 16H); 1.82(d, 3H, J=6Hz); | 0.90-1.97(m, 16H); 2.27(d, 3H, J=6Hz); |
| | 4.60(m, 1H); 6.42(q, 1H, J=6Hz) | 4.64(m, 1H); 6.79(q, 1H, J=6Hz) |
| 8′ | see ex. 6 | see ex. 6 |
| 9′ | see ex. 7 | see ex. 7 |
| 10′ | 1.87(d, 3H, J=6Hz); 4.28(t, 1H, J=7Hz); | 2.28(d, 3H, J=6Hz); 4.28(t, 1H, J=7Hz); |
| | 4.42(dd, 1H, J=7Hz, 10Hz); 4.52(dd, 1H, J=7Hz, 10Hz); | 4.40(dd, 1H, J=7Hz, 10Hz); 4.53(dd, 1H, J=7Hz, 10Hz); |
| | 6.46(q, 1H, J=6Hz); 7.32(m, 2H); 7.42(m, 2H); | 6.70(q, 1H, J=6Hz); 7.32(m, 2H); 7.42(m, 2H); |
| | 7.61(m, 2H); 7.77(m, 2H) | 7.61(m, 2H); 7.78(m, 2H) |
| 11′ | 0.78/0.81(sxd, 3H, J=6Hz); 1.93/1.94(2xd, 3H, J=6Hz); | 0.76/0.89(sxd, 3H, J=6Hz); 2.21/2.23(2xd, 3H, J=6Hz); |
| | 4.39/4.43(sxd, 1H, J=4Hz); 5.57(m, 1H); 6.55/6.56(2xq, 1H, | 4.379/4.40(2xd, 1H, J=4Hz); 5.56(m, 1H); |
| | J=6Hz); 7.34(m, 2H); 7.42(m, 2H); 7.57(m, 1H); | 6.86/6.88(2xq, 1H, J=6Hz); 7.30(m, 2H); |
| | 7.72(m, 1H); 7.79(m, 2H) | 7.40(m, 2H); 7.52(m, 1H); 7.70(m, 1H); 7.73(m, 2H) |

For intermediates of EX12` to example 24` see Examples 1a, 1b and Example 10.

The intermediate numbering indicated with “X`” corresponds to the Example number “X” in TABLE 1.

3-{(E)[[1-trans-(4-Amino-cyclohexylamino)-iminomethyl]-methylhydrazono]methyl}-7-{[(5-amino-1,2,4-thiadiazol-3-yl)-(Z)-(fluoromethoxyimino)-acetyl]amino}-3-cephem-4-carboxylic acid

a) Benzylidene derivative of 3-amino-1-(trans-4-aminocyclohexyl)-3-methyl-guanidine

35 g of the benzylidene derivative of S-methyl-2-methyl-isothiosemicarbazide in the form of a hydrochloride and 32.79 g of trans-1,4-diaminocyclohexane in 300 ml of MeOH are refluxed. The mixture obtained is stirred at RT, a precipitate formed is filtered off and solvent is evaporated. The evaporation residue obtained is treated with 217.5 ml of 2M HCl, a precipitate formed is filtered off, washed and dried. The volume of the filtrate obtained is brought to about 150 ml, a precipitate is formed is filtered off, washed and dried. The dried, combined precipitates are recristallized from H₂O and the benzylidene derivative of 3-amino-1-(trans-4-aminocyclo hexyl)-3-methyl-guanidine in the form of a monohydrochloride is obtained.

b) 3-Amino-1-(trans-4-aminocyclohexyl)-3-methyl-guanidine

From a mixture of 24.74 g of benzylidene derivative of 3-amino-1-(trans-4-aminocyclohexyl)-3-methyl-guanidine in the form of a monohydrochloride in 79.9 ml of 2M HCl, benzaldehyde is destined off and solvent from the remaining mixture is evaporated. 3-Amino-1-(trans-4-aminocyclohexyl)-3-methyl-guanidine in the form of a dihydrochloride is obtained.

c) 3-{(E)[[-trans-(4-amino-cyclohexylamino)-iminomethyl]-methylhydrazono]methyl}-7-{[(5-amino-[1,2,4]thiadiazol-3-yl)-(Z)-(fluoromethoxyimino)-acetyl]amino}-3-cephem-4-carboxylic acid

2.78 g of N-(1,4,5a,6-tetrahydro-3-hydroxy-1,7-dioxo-3H,7H-azeto(2,1-b)furo(3,4-d)(1,3)-thiazin-6-yl)-2-(5-amino-1,2,4-thiadiazol-3-yl)-(Z)-2-(fluoromethoxyimino) acetic acid amide are added to a mixture of 2 g of 3-amino-1-(trans-4-aminocyclohexyl)-3-methyl-guanidine in the form of a dihydrochloride in 3.4 ml of 2M HCl and 6.1 ml of DMA and the suspension obtained is stirred at RT. The mixture obtained is poured into CH₃CN under stirring. A precipitate formed is filtrated off, washed and dried. 3-{(E)[[1-trans-(4-Amino-cyclohexylamino)-iminomethyl]-methylhydrazono]methyl}-7-{[(5-amino-1,2,4-thiadiazol-3-yl)-(Z)-(fluoromethoxyimino)-acetyl]amino}-cephem-4-carboxylic acid in the form of a trihydrochloride is obtained.

d) 3-{(E)[[1-trans-(4-Amino-cyclohexylamino)-iminomethyl]-methylhydrazono]methyl}-7-{[(5-amino-1,2,4-thiadiazol-3-yl)-(Z)-(fluoromethoxyimino)-acetyl]amino}-3-cephem-4-carboxylic acid

10 g of crude 3-{(E)[[1-trans-(4-amino-cyclohexylamino)-iminomethyl]-methylhydrazono]methyl}-7-{[(5-amino-1,2,4-thiadiazol-3-yl)-(Z)-(fluoromethoxyimino)-acetyl]amino}-3-cephem-4-carboxylic acid in the form of a trihydrochloride are dissolved in 42 ml of H₂O and subjected to chromatography (LiChroprep® RP-18, Merck, grain size 40-63 μm). Fractions containing the desired product in the form of a monohydrochloride are combined and optionally lyophilised. 3-{(E)[[1-Trans-(4-amino-cyclohexylamino)-iminomethyl]-methylhydrazono]methyl}-7-{([(5-amino-1,2,4-thiadiazol-3-yl)-(Z)-(fluoromethoxyimino)-acetyl]amino}-3-cephem-4-carboxylic acid in the form of a hydrochloride is obtained.

¹H-NMR: 1.30-1.70, m, 4H, CCH₂; 1.80-2.10, m, 4H, CCH₂; 2.88-3.10, m, 1H, NCH; 3.32, s, 3H, NCH₃; 3.42-3.70, m, 2H, 1H from SCH₂ and 1H from NCH; 4.25, part of the AB-quartet, J=18 Hz, 1H, SCH₂; 5.28, d, J=5 Hz, 1H, β-lactam; 5.79, d, J=55 Hz, 2H, CH₂F; 5,75, dd, J=5 Hz and 8 Hz, 1H, β-lactam; 8.10, s, 1H, CH═N; 9.84, d, J=8 Hz, 1H, NH.

1. A pharmaceutically active compound having a carboxylic acid group —COOH as a part of its chemical structure which —COOH group is in the form of a carboxylic acid ester and which carboxylic acid ester is selected from the group consisting of
1-(2,3-disubstituted 1-propoxycarbonyloxy)-ethyl carboxylic acid ester, wherein the substituents are selected from the group consisting of hydroxy and (C₁₋₂₂)alkylcarbonyloxy,
1-(1,3-disubstituted 2-propoxycarbonyloxy)-ethyl carboxylic acid ester, wherein the substituents are selected from the group consisting of hydroxy and (C₁₋₂₂)alkylcarbonyloxy,
1-(9H-fluorene-9-yl-(C₁₋₄)alkanyloxycarbonyloxy)-ethyl carboxylic acid ester,
1-(decahydro-naphthalene-2-yl-oxycarbonyloxy)-ethyl carboxylic acid ester and
1-(2-amino(C₁₋₆)alkoxycarbonyloxy)-ethyl carboxylic acid ester,
with the proviso that, if the pharmaceutically active compound is a penicillin, then, 1-(2-amino(C₁₋₄)alkoxycarbonyloxy)-ethyl carboxylic acid esters are excluded.

2. A compound of claim 1, wherein the pharmaceutically active compound is a β-lactam antibiotic.

3. A compound of claim 1, wherein the pharmaceutically active compound is a cephalosporin, having the formula 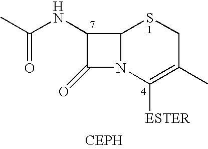
wherein ESTER is a carboxylic acid ester as defined in claim 1.

4. A compound of claim 1, wherein the pharmaceutically active compound is a penicillin, having the formula 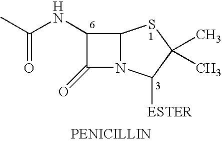
and ESTER is a carboxylic acid ester as defined in claim 1.

5. A compound of claim 1, wherein the —COOH group is in the form of an ester selected from the group consisting of
1-(2,3,-dihydroxy-1-propoxycarbonyloxy)-ethyl-oxy-carbonyl,
1-(2,3-diacetoxy-1-propoxycarbonyloxy)-ethyl-oxy-carbonyl,
1-(1,3-diacetoxy-2-propoxycarbonyloxy)-ethyl-oxy-carbonyl,
1-(2-octanoyloxy-3-acetoxy-1-propoxycarbonyloxy)-ethyl-oxy-carbonyl,
1-(2-acetoxy-3-octanoyloxy-1-propoxycarbonyloxy)-ethyl-oxy-carbonyl,
1-(9H-fluorene-9-yl-methoxycarbonyloxy)-ethyl-oxy-carbonyl,
1-(9H-fluorene-9-yl-ethoxycarbonyloxy)-ethyl-oxy-carbonyl,
1-(decahydro-naphthalene-2-yl-oxycarbonyloxy)-ethyl-oxy-carbonyl and
1-(2-amino-ethoxycarbonyloxy)-ethyl-oxy-carbonyl, with the proviso of claim 1.

6. A compound of claim 3, wherein the cephalosporin of formula CEPH is selected from the group consisting of
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-{[3-ethyl-2-(methylimino)-imidazolidin-1-yl]-hydrazonomethyl}-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-[(carbamimidoyl)-hydrazonomethyl]-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-[(1,4,5,6,-tetrahydropyrimidin-2-yl)-hydrazonomethyl]-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-{methyl-[N-(4-amino-cyclohexyl)-carbamimidoyl]-hydrazonomethyl}-8-oxo-5-thia-1-aza-bicyclo[4.2.0]oct-2-ene-2-carboxylic acid,
7-{2-(5-Amino-[1,2,4]thiadiazol-3-yl)-2-(fluoromethoxyimino)-acetylamino}-3-[(2-dimethylamino-4,5-dihydro-imidazol-1-yl-imino)-methyl]-8-oxo-5-thia-1-aza-bicyclo[4.2.0]oct-2-ene-2-carboxylic acid,
3-Carbamoyloxymethyl-7-{2-furan-2-yl-[(Z)-methoxyimino]-acetylamino}-8-oxo-5-thia-1-aza-bicyclo[4.2.0]oct-2-ene-2-carboxylic acid, and
7-{2-(5-Amino-[1,2,4]thiadiazol-3-yl)-2-(fluoromethoxyimino)-acetylamino}-3-[(3-ethyl-2-(methylimino)-imidazol-1-yl-imino)-methyl]-8-oxo-5-thia-1-aza-bicyclo[4.2.0]oct-2-ene-2-carboxylic acid,

7. A compound of claim 1, selected from the group consisting of
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,3-diacetoxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(1,3-diacetoxy-2-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,-octanoyl-3-acetoxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2-acetoxy-3-octanoyl-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2-aminoethoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(decahydro-naphthalen-2-yl-oxycarbonyloxy)-ethyl ester,
7-{2-(5-Amino-[1,2,4]thiadiazol-3-yl)-2-(fluoromethoxyimino)-acetylamino}-3-[(imino-piperazin-1-yl-methyl)-hydrazonomethyl]-8-oxo-5-thia-1-aza-bicyclo[4.2.0]oct-2-ene-2-carboxylic acid 1-(2-aminoethoxycarbonyloxy)-ethyl ester,
7-{2-(5-Amino-[1,2,4]thiadiazol-3-yl)-2-(fluoromethoxyimino)-acetylamino}-3-[(imino-piperazin-1-yl-methyl)-hydrazonomethyl]-8-oxo-5-thia-1-aza-bicyclo[4.2.0]oct-2-ene-2-carboxylic acid 1-(decahydro-naphthalen-2-yl-oxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-([1-(9H-fluoren-9-yl)-ethoxycarbonyloxy)-ethyl ester,
7-{2-(5-Amino-[1,2,4]thiadiazol-3-yl)-2-(fluoromethoxyimino)-acetylamino}-3-[{[3-ethyl-2-(methylimino)-imidazolidin-1-yl]-hydrazonomethyl}-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-[carbamimidoyl)-hydrazonomethyl]-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-[(1,4,5,6,-tetrahydropyrimidin-2-yl)-hydrazonomethyl]-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-{methyl-[N-(4-amino-cyclohexyl)-carbamimidoyl]-hydrazonomethyl}-8-oxo-5-thia-1-aza-bicyclo[4.2.0]oct-2-ene-2-carboxylic acid 1-(9H-fluoren-9-yl-methoxycarbonyloxy)-ethyl ester,
7-{2-(5-Amino-[1,2,4]thiadiazol-3-yl)-2-(fluoromethoxyimino)-acetylamino}-3-[(2-dimethylamino-4,5-dihydro-imidazol-1-yl-imino)-methyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-y)-2-(fluoromethoxyimino)-acetylamino}-3-[(carbamimidoyl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[(1,4,5,6,-tetrahydropyrimidin-2-yl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[(2-dimethylamino-4,5-dihydro-imidazol-1-yl-imino)-methyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-[methyl-(imino-piperazin-1-yl-methyl)-hydrazonomethyl]-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-{[3-ethyl-2-(methylimino)-imidazolidin-1-yl]-hydrazonomethyl}-8-oxo-5-thia-1-aza-bicyclo[4.2.0]oct-2-ene-2-carboxylic acid 1-(2,3-dihydroxy-1-propoxycarbonyloxy)-ethyl ester,
7-{2-(5-Amino-[1,2,4]thiadiazol-3-yl)-2-(fluoromethoxyimino)-acetylamino}-3-{methyl-[N-(4-amino-cyclohexyl)-carbamimidoyl]-hydrazonomethyl}-8-oxo-5-thia-1-aza-bicyclo[4.2.0]oct-2-ene-2-carboxylic acid 1-(2,3-dihydroxy-1-propoxycarbonyloxy)-ethyl ester,
3-Carbamoyloxymethyl-7-{2-furan-2-yl-[(Z)-methoxyimino]-acetylamino}-8-oxo-5-thia-1-aza-bicyclo[4.2.0]oct-2-ene-2-carboxylic acid 1-(9H-fluoren-9-yl-methoxycarbonyloxy)-ethyl ester, and
3-Carbamoyloxymethyl-7-{2-furan-2-yl-[(Z)-methoxyimino]-acetylamino}-8-oxo-5-thia-1-aza-bicyclo[4.2.0]oct-2-ene-2-carboxylic acid 1-(2,3-dihydroxy-1-propoxycarbonyloxy)-ethyl ester,

8. A compound of claim 1 in the form of a salt.

9. A pharmaceutical composition comprising a compound of claim 1.

10. A method for treating a microbial disease comprising administering to a subject in need thereof a compound of claim 1.

11. A pharmaceutical composition comprising a compound of claim 1 and at least one pharmaceutical excipient.

12. A pharmaceutical composition comprising a compound of claim 1 and another pharmaceutically active compound.

13. (canceled)

14. A method for treatment of microbial diseases in a subject in need of such treatment, which method comprises administering to said subject an effective amount of a compound of claim 1.

15. A method for treating a microbial disease comprising administering to a subject in need thereof a compound of claim 1 concomitantly or in sequence with at least one immunosuppressant, immunomodulatory or anti-inflammatory active agent.
